(19) United States
(12) Patent Application Publication
(10) Pub. No.: US 2024/0336678 A1
(43) Pub. Date: Oct. 10, 2024

(54) ANTI-IL-13 ANTIBODY FORMULATION

(71) Applicant: Medlmmune LLC, Gaithersburg, MD (US)

(72) Inventors: Ramin SAMADANI, Gaithersburg, MD (US); Sureshkumar Bhanaram Choudhary, Gaithersburg, MD (US)

(21) Appl. No.: 18/681,969

(22) PCT Filed: Aug. 16, 2022
(86) PCT No.: PCT/US2022/074992

§ 371 Date: Feb. 7, 2024

Related U.S. Application Data
(60) Provisional application No. 63/233,440, filed on Aug. 16, 2021.

(51) Int. Cl. C07K 16/24; A61K 47/18; A61K 47/22; A61K 47/26

(52) U.S. Cl. CPC C07K 16/244; A61K 47/183; A61K 47/22; A61K 47/26; C07K 2317/21; C07K 2317/622; C07K 2317/624

(57) ABSTRACT

The present invention relates to a pharmaceutical composition comprising an anti-IL-13 antibody or an IL-13-binding fragment thereof, wherein the pharmaceutical composition comprises histidine buffer, a positively charged-excipient (e.g. lysine or arginine) and a surfactant. Such pharmaceutical compositions may be used for treating an IL-13 related disorder, such as atopic dermatitis, asthma, allergic rhinitis, fibrosis, chronic obstructive pulmonary disease, scleroderma, inflammatory bowel disease or Hodgkin's lymphoma.

### FIELD OF THE INVENTION

The present invention relates to a pharmaceutical composition comprising an anti-IL-13 antibody or an IL-13-binding fragment thereof, wherein the pharmaceutical composition comprises histidine buffer, a positively charged-excipient (e.g. lysine or arginine) and a surfactant. Such pharmaceutical compositions may be used for treating an IL-13 related disorder, such as atopic dermatitis, asthma, allergic rhinitis, fibrosis, chronic obstructive pulmonary disease, scleroderma, inflammatory bowel disease or Hodgkin's lymphoma.

### BACKGROUND

IL-13 is a 114 amino acid cytokine with an unmodified molecular mass of approximately 12 kDa. IL-13 is most closely related to IL-4 with which it shares 30% sequence homology at the amino acid level. The human IL-13 gene is located on chromosome 5q31 adjacent to the IL-4 gene. Although initially identified as a Th2 CD4+ lymphocyte derived cytokine, IL-13 is also produced by Th1 CD4+ T-cells, CD8+ T lymphocytes NK cells, and non-T-cell populations such as mast cells, basophils, eosinophils, macrophages, monocytes and airway smooth muscle cells. IL-13 has been linked with a number of diseases, in particular, diseases which are caused by an inflammatory response. For example, administration of recombinant IL-13 to the airways of naive non-sensitised rodents was shown to cause many aspects of the asthma phenotype including airway inflammation, mucus production and airways hyper-responsiveness (Wills-Karp, M. et al., Science, 1998. 282 (5397), 2258-2261; Grunig, G. et al., Science, 1998. 282 (5397), 2261-2263; Venkayya, R., et al., Am J Respir Cell Mol Biol, 2002. 26 (2), 202-208; Morse, B. et al., Am J Physiol Lung Cell Mol Physiol, 2002. 282 (1), L44-49). A similar phenotype was observed in a transgenic mouse in which IL-13 was specifically overexpressed in the lung. In this model, more chronic exposure to IL-13 also resulted in fibrosis (Zhu, Z. et al., J Clin Invest, 1999. 103 (6), 779-788).

A number of genetic polymorphisms in the IL-13 gene have also been linked to allergic diseases. In particular, a variant of the IL-13 gene in which the arginine residue at amino acid 130 is substituted with glutamine (Q130R) has been associated with bronchial asthma, atopic dermatitis and raised serum IgE levels (Heinzmann, A. et al., Hum Mol Genet, 2000. 9 (4), 549-559; Howard, T. D. et al., Am J Hum Genet, 2002. 70 (1), 230-236; Kauppi, P. et al., Genomics, 2001. 77 (1-2), 35-42; Graves, P. E. et al., J Allergy Clin Immunol, 2000. 105 (3), 506-513). This particular IL-13 variant is also referred to as the Q110R variant (arginine residue at amino acid 110 is substituted with glutamine) by some groups who exclude the 20 amino acid signal sequence from the amino acid count.

Upregulation of IL-13 and interleukin-4 (IL-4) in lesional and nonlesional skin is a key feature of atopic dermatitis (AD), suggesting both cytokines can contribute to AD pathogenesis (see Nomura, I., Goleva, E., Howell, M. D., Hamid, Q. A., Ong, P. Y., Hall, C. F. et al. Cytokine milieu of atopic dermatitis, as compared to psoriasis, skin prevents induction of innate immune response genes. J Immunol. 2003; 171:3262-3269; Tazawa, T., Sugiura, H., Sugiura, Y., and Uehara, M. Relative importance of IL-4 and IL-13 in lesional skin of atopic dermatitis. Arch Dermatol Res. 2004; 295:459-464). Moreover, AD severity is associated with increased IL-13 and associated chemokine mRNA and serum levels, whereas reductions in IL-13 concentrations have correlated with treatment response and improved clinical outcomes.

IL-13 production has also been associated with allergic asthma (van der Pouw Kraan, T. C. et al., Genes Immun, 1999. 1 (1), 61-65) and raised levels of 15 IL-13 have been measured in human subjects with atopic rhinitis (hay fever), allergic dermatitis (eczema) and chronic sinusitis.

Aside from asthma, IL-13 has been associated with other fibrotic conditions. Increased levels of IL-13, up to a 1000 fold higher than IL-4, have been 20 measured in the serum of patients with systemic sclerosis and in bronchoalveolar lavage (BAL) samples from patients affected with other forms of pulmonary fibrosis (Hasegawa, M. et al., J Rheumatol, 1997. 24 (2), 328-332; Hancock, A. et al., Am J Respir Cell Mol Biol, 1998. 18 (1), 60-65).

It has been demonstrated that overexpression of IL-13 in the mouse lung caused emphysema, elevated mucus production and inflammation, reflecting aspects of human chronic obstructive pulmonary disease (COPD) (Zheng, T. et al., J Clin Invest, 2000. 106 (9), 1081-1093).

It has been proposed that IL-13 may also play a role in the pathogenesis of inflammatory bowel disease (Heller, F. et al., Immunity, 2002. 17 (5), 629-38) and raised levels of IL-13 have been detected in the serum of some Hodgkin's disease patients when compared to normal controls (Fiumara, P. et al., Blood, 2001. 98 (9), 2877-2878).

IL-13 inhibitors are also believed to be therapeutically useful in the prevention of tumour recurrence or metastasis (Terabe, M. et al., Nat Immunol, 2000. 1 (6), 515-520). Inhibition of IL-13 has also been shown to enhance anti-viral vaccines in animal models and may be beneficial in the treatment of HIV and other infectious diseases (Ahlers, J. D. et al., Proc Natl Acad Sci USA, 2002. 99 (20), 13020-13025).

An antibody directed approach to IL-13 inhibition has been described. For example, WO 2005/007699 describes a series of human anti-IL-13 antibody molecules which are shown to neutralise IL-13 activity and which are claimed to be of potential use in the treatment of IL-13 related disorders. WO 2007/036745 describes a pharmaceutical composition comprising an anti-IL-13 antibody and its use to treat IL-13 related disorders.

Tralokinumab (also known as CAT-354 and BAK502G9) is a fully human therapeutic antibody that binds to and neutralizes IL-13, including the Q130R variant (see Popovic et al. J. Mol. Biol. (2017) 429 (2): 208-219; May, R. D., Monk, P. D., Cohen, E. S., Manuel, D., Dempsey, F., Davis, N. H. et al. Preclinical development of CAT-354, an IL-13 neutralizing antibody, for the treatment of severe uncontrolled asthma. Br J Pharmacol. 2012; 166:177-193).

Tralokinumab has previously been tested in phase 2b study of 204 adults for the treatment of AD—where patients received 45 mg, 150 mg, or 300 mg of subcutaneous tralokinumab, or placebo, every 2 weeks for 12 weeks with concomitant topical glucocorticoids—and was found to improve change from baseline in Eczema Area Severity Index (EASI) score, together with improvements in Scoring atopic dermatitis (SCORAD), Dermatology Life Quality Index (DLQI), and pruritus numeric rating scale scores, as compared to placebo (Wollenberg J. Allergy Clin. Immunol. (2019) 143 (1): 135-141).

There remains a desire in the art for further and improved pharmaceutical compositions comprising an anti-IL-13 antibody, particularly those that comprise high concentrations of an anti-IL-13 antibody. One of the major challenges for developing such pharmaceutical compositions is the high viscosity of anti-IL13 antibodies, such as Tralokinumab, at high concentrations, e.g. 150 mg/mL. The present invention has been devised in light of such considerations.

### SUMMARY OF THE INVENTION

The invention relates to pharmaceutical compositions comprising an anti-IL-13 antibody or an IL-13-binding fragment thereof, which have been shown by the inventors to have decreased viscosity and reduced sensitivity to visible light compared to a known pharmaceutical composition comprising an anti-IL-13 antibody.

The pharmaceutical compositions disclosed herein comprise an anti-IL-13 antibody or an IL-13-binding fragment thereof, a histidine buffer, a positively charged excipient, and a surfactant, pH 5.2-5.7.

In one aspect, the invention provides a pharmaceutical composition comprising an anti-IL-13 antibody or an IL-13-binding fragment thereof, 20 mM±40% histidine buffer, 150 mM±40% a positively charged excipient (e.g. lysine or arginine), 0.01% surfactant (e.g. polysorbate 80, pH 5.5±0.1).

In a preferred embodiment, the pharmaceutical composition comprises an anti-IL-13 antibody or an IL-13-binding fragment thereof, 15-25 mM histidine buffer, 100-200 mM lysine or arginine, 0.01% polysorbate 80, pH 5.5±0.1. Most preferably, the histidine buffer is present in an amount of 20 mM±10% and lysine or arginine is present in an amount of 150 mM±10%. In a preferred embodiment, the positively charged excipient is lysine.

In a preferred embodiment, the anti-IL-13 antibody or IL-13-binding fragment thereof is present at a concentration of between 130 and 170 mg/ml. Most preferably, the anti-IL-13 antibody or IL-13-binding fragment thereof is present at a concentration of 150 mg/ml±10%.

In a preferred embodiment, the anti-IL-13 antibody or IL-13-binding fragment thereof comprises a heavy chain variable region (HCVR) and a light chain variable region (LCVR), wherein:
(i) the heavy chain variable region comprises:
   a heavy chain complementarity determining region 1 (HCDR1) comprising an amino acid sequence of SEQ ID NO:1;
   a heavy chain complementarity determining region 2 (HCDR2) comprising an amino acid sequence of SEQ ID NO:2; and
   a heavy chain complementarity determining region 3 (HCDR3) comprising an amino acid sequence of SEQ ID NO:3; and
(ii) the light chain variable region comprises:
   a light chain complementarity determining region 1 (LCDR1) comprising an amino acid sequence of SEQ ID NO:4;
   a light chain complementarity determining region 2 (LCDR2) comprising an amino acid sequence of SEQ ID NO:5; and
   a light chain complementarity determining region 3 (LCDR3) comprising an amino acid sequence of SEQ ID NO:6.

Preferably, the anti-IL-13 antibody or IL-13-binding fragment thereof comprises a heavy chain variable region sequence of SEQ ID NO: 8 and a light chain variable region sequence of SEQ ID NO: 10. More preferably, the anti-IL-13 antibody or IL-13-binding fragment thereof comprises a heavy chain sequence of SEQ ID NO: 11 and a light chain sequence of SEQ ID NO: 12. Most preferably, the anti-IL-13 antibody is Tralokinumab.

In another aspect, the invention provides a pharmaceutical composition as described in any of the aspects and embodiments disclosed herein for use in a method for treatment of an IL-13 related disorder.

In a further aspect, the invention provides a method for the treatment of an IL-13 related disorder, wherein the method comprises the step of administering the pharmaceutical composition as described in any of the aspects and embodiments disclosed herein in a therapeutically effective amount to a patient in need thereof.

In yet a further aspect, the invention provides use of a pharmaceutical composition as described in any of the aspects and embodiments disclosed herein in the manufacture of a medicament for the treatment of an IL-13 related disorder.

In any of these aspects, the IL-13 related disorder may be atopic dermatitis, asthma, allergic rhinitis, fibrosis, chronic obstructive pulmonary disease, scleroderma, inflammatory bowel disease or Hodgkin's lymphoma, for example. In a preferred embodiment, the IL-13 related disorder is atopic dermatitis.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows the viscosity of five Tralokinumab formulations tested against the current formulation (Formulation A). Formulation A comprises 150 mg/ml Tralokinumab with 50 mM sodium acetate/acetic acid, 85 mM sodium chloride, and 0.01% w/v polysorbate 80, at pH 5.5. The tested formulations include (i) 150 mM proline and 100 mM sucrose, (ii) 150 mM glutamate, (iii) 150 mM NaCl, (iv) 150 mM arginine or (v) 150 mM lysine. Viscosity was measured over a range of temperatures.

FIG. 2 shows viscosity of a formulation comprising Tralokinumab and 20 mM histidine buffer, 150 mM lysine and 0.01% w/v polysorbate 80, at pH 5.5 (Formulation B) compared with viscosity of Formulation A. Tralokinumab concentrations between 130-170 mg/ml were used in both formulations. Viscosity was measured at (A) 18° C. or (B) 23° C.

FIG. 3 shows the viscosity of Formulation B (20 mM histidine buffer, 150 mM lysine and 0.01% w/v polysorbate 80, at pH 5.5) compared to Formulation A (50 mM sodium acetate/acetic acid, 85 mM sodium chloride, and 0.01% w/v polysorbate 80, at pH 5.5) at various temperatures between 5-30° C. Tralokinumab was used at a concentration of 170 mg/mL.

FIG. 4 shows (A) the force required to expel Formulations A or B from prefilled syringes (glide force) for varying instron rates and (B) the injection durations vs glide force for Formulations A and B. Tralokinumab was used at a concentration of 168 mg/mL.

FIG. 5A shows large volume bolus injector (LVBI) infusion duration versus viscosity. The dotted lines corresponding to Formulations A and B are labelled. The duration of infusion and viscosities for various Tralokinumab concentrations for (b) Formulation A and (c) Formulation B are also shown.

FIG. 6 shows the stability of Formulation B in 1 mL prefilled syringe compared to Formulation A. Tralokinumab was used at a concentration of 168 mg/mL. The stability was compared at (a) 5° C. and (b) 25° C. Purity was measured at various time points by size exclusion chromatography (SEC).

FIG. 7 shows the effect of addition of lysine or arginine on the levels of (a) subvisible or (b) visible particles in Formulation A after 3 days at 5° C. without any polysorbate-80 (PS80).

FIG. 8 shows the sensitivity of Formulation B to light exposure compared to Formulation A. Aggregates were measured by size exclusion chromatography (SEC). A Tralokinumab concentration of 170 mg/ml was used.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The invention relates to pharmaceutical compositions comprising an anti-IL-13 antibody or an IL-13-binding fragment thereof, wherein the pharmaceutical composition comprises histidine buffer and lysine, as well as to the use of these pharmaceutical compositions to treat IL-13 related disorders.

### Anti-IL-13 Antibodies and IL-13-Binding Fragments Thereof

The term “antibody”, as used herein, includes immunoglobulin molecules comprising four polypeptide chains, two heavy (H) chains and two light (L) chains inter-connected by disulfide bonds, as well as multimers thereof (e.g. IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some cases, the FRs of the anti-IL-13 antibody (or IL-13-binding fragment or derivative thereof) may be identical to the human germline sequences, or may be naturally or artificially modified.

The heavy chain constant region of the antibodies may be from any types of constant region, such as IgG, IgM, IgD, IgA, and IgE. Generally, the antibody is an IgG (e.g. isotype IgG1, IgG2, IgG3 or IgG4). Preferably, the antibody is an IgG4, as exemplified herein.

The antibody may be a mouse, human, primate, humanized or chimeric antibody. The antibody may be polyclonal or monoclonal. For therapeutic applications, monoclonal and human (or humanized) antibodies are preferred. In a particularly preferred embodiment, the antibody is human or humanized, and monoclonal.

The antibody can be a multispecific (e.g. bispecific) antibody. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen binding fragment of an antibody as described herein using routine techniques available in the art. For example, the methods that use of bispecific antibodies, wherein one arm of an immunoglobulin is specific for IL-13, and the other arm of the immunoglobulin is specific for a second therapeutic target or is conjugated to a therapeutic moiety.

An IL-13-binding fragment of an anti-IL-13 antibody may be any naturally occurring, enzymatically obtainable, synthetic, or genetically engineered polypeptide. Such fragments may be derived, e.g. from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g. commercial sources, DNA libraries (including, e.g. phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of IL-13-binding fragments include: Fab, Fab′, F(ab′)2, Fd, Fv, single-chain Fv (scFv), disulphide-linked Fvs, dAb fragments, and other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains.

An IL-13-binding fragment of an anti-IL-13 antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

The anti-IL-13 antibody, or an IL-13-binding fragment thereof, may comprise: a heavy chain complementarity determining region 1 (HCDR1) comprising an amino acid sequence of SEQ ID NO:1; a heavy chain complementarity determining region 2 (HCDR2) comprising an amino acid sequence of SEQ ID NO: 2; a heavy chain complementarity determining region 3 (HCDR3) comprising an amino acid sequence of SEQ ID NO:3; a light chain complementarity determining region 1 (LCDR1) comprising an amino acid sequence of SEQ ID NO:4; a light chain complementarity determining region 2 (LCDR2) comprising an amino acid sequence of SEQ ID NO:5; and a light chain complementarity determining region 3 (LCDR3) comprising an amino acid sequence of SEQ ID NO:6.

The anti-IL-13 antibody, or an IL-13-binding fragment thereof, may comprise a heavy chain variable region (HCVR) and a light chain variable region (LCVR), wherein: (i) the heavy chain variable region comprises: a heavy chain complementarity determining region 1 (HCDR1) comprising an amino acid sequence of SEQ ID NO: 1; a heavy chain complementarity determining region 2 (HCDR2) comprising an amino acid sequence of SEQ ID NO:2; and a heavy chain complementarity determining region 3 (HCDR3) comprising an amino acid sequence of SEQ ID NO:3; and (ii) the light chain variable region comprises: a light chain complementarity determining region 1 (LCDR1) comprising an amino acid sequence of SEQ ID NO:4; a light chain complementarity determining region 2 (LCDR2) comprising an amino acid sequence of SEQ ID NO: 5; and a light chain complementarity determining region 3 (LCDR3) comprising an amino acid sequence of SEQ ID NO:6.

In addition, the anti-IL-13 antibody, or an IL-13-binding fragment thereof, may comprise: (i) an amino acid sequence that is at least 80%, at least 85%, at least 90%, at least 95%, at least 96%, at least 97%, at least 98%, or at least 99% identical to a heavy chain variable region sequence of SEQ ID NO: 8; and/or (ii) an amino acid sequence that is 80%, at least 85%, at least 90%, at least 95%, at least 96%, at least 97%, at least 98%, or at least 99% identical to a light chain variable region sequence of SEQ ID NO: 10. The anti-IL-13 antibody, or an IL-13-binding fragment thereof, may comprise a heavy chain variable region sequence of SEQ ID NO: 8 and a light chain variable region sequence of SEQ ID NO: 10.

The anti-IL-13 antibody, or the IL-13-binding fragment thereof, may comprise: (i) an amino acid sequence that is at least 80%, at least 85%, at least 90%, at least 95%, at least 96%, at least 97%, at least 98%, or at least 99% identical to the heavy chain sequence of SEQ ID NO: 11; and/or (ii) an amino acid sequence that is at least 80%, at least 85%, at least 90%, at least 95%, at least 96%, at least 97%, at least 98%, or at least 99% identical to the light chain sequence of SEQ ID NO: 12. In some cases, the anti-IL-13 antibody, or an IL-13-binding fragment or IL-13-binding derivative thereof, comprises a heavy chain of SEQ ID NO: 11 and a light chain sequence of SEQ ID NO: 12.

One such antibody that can be used in the methods described herein is the anti-IL-13 antibody, tralokinumab (as described in the “International Nonproprietary Names for Pharmaceutical Substances (INN)” list 102 (WHO Drug Information (2009) 23 (4): pp 348)). Tralokinumab is a fully human IgG4-lambda antibody, which specifically binds and neutralises human IL-13.

TABLE 1
Tralokinumab

| SEQ ID number | Name | Sequence |
|---|---|---|
| SEQ ID NO: 1 | HCDR1 | NYGLS |
| SEQ ID NO: 2 | HCDR2 | WISANNGDTNYGQEFQG |
| SEQ ID NO: 3 | HCDR3 | DSSSSWARWFFDL |
| SEQ ID NO: 4 | LCDR1 | GGNIIGSKLVH |
| SEQ ID NO: 5 | LCDR2 | DDGDRPS |
| SEQ ID NO: 6 | LCDR3 | QVWDTGSDPVV |
| SEQ ID NO: 7 | cDNA heavy | caggtccagctggtgcagtctggggctgaggtgaagaagcctggggcctca |
| | chain | gtgaaggtctcctgcaaggcttctggttacacctttacaaattatggtctc |
| | variable | agctgggtgcgacaggcccctggacaagggcttgagtggatgggatggat |
| | domain | cagcgctaataatggcgacacaaattatggacaggaattccagggcaga |
| | | gtcaccatgaccacagatacatccacgagcacagcctacatggagttga |
| | | ggagcctgagatctgacgacacggccgtttattactgtgcgagagactcca |
| | | gcagcagctgggcccgctggtttttcgatctctggggccgggggacactgg |
| | | tcaccgtctcctca |
| SEQ ID NO: 8 | polypeptide | QVQLVQSGAEVKKPGASVKVSCKASGYTFTNYGLS |
| | sequence | WVRQAPGQGLEWMGWISANNGDTNYGQEFQGRVT |
| | heavy chain | MTTDTSTSTAYMELRSLRSDDTAVYYCARDSSSSWA |
| | variable | RWFFDLWGRGTLVTVSS |
| | region | |
| SEQ ID NO: 9 | cDNA light | tcctatgtgctgactcagccaccctcggtgtcagtggccccaggaaagac |
| | chain | ggccaggattacctgtgggggaaacatcattggaagtaaacttgtacactg |
| | variable | gtaccagcagaagccaggccaggcccctgtgctggtcatctatgatgatg |
| | domain | gcgaccggccctcagggatccctgagcgattctctggctccaactctggga |
| | | acacggccaccctgaccatcagcagggtcgaggccggggatgaggcc |
| | | gactattattgtcaggtgtgggatactggtagtgatcccgtggtattcggc |
| | | ggagggaccaagctgaccgtcctaggt |
| SEQ ID NO: 10 | polypeptide | SYVLTQPPSVSVAPGKTARITCGGNIIGSKLVHWYQQ |
| | sequence | KPGQAPVLVIYDDGDRPSGIPERFSGSNSGNTATLTI |
| | light | SRVEAGDEADYYCQVWDTGSDPVVFGGGTKLTVL |
| | chain | |
| | variable | |
| | region | |
| SEQ ID NO: 11 | Heavy chain | QVQLVQSGAEVKKPGASVKVSCKASGYTFTNYGLS |
| | | WVRQAPGQGLEWMGWISANNGDTNYGQEFQGRVT |
| | | MTTDTSTSTAYMELRSLRSDDTAVYYCARDSSSSWA |
| | | RWFFDLWGRGTLVTVSSASTKGPSVFPLAPCSRSTS |
| | | ESTAALGCLVKDYFPEPVTVSWNSGALTSGVHTFPA |
| | | VLQSSGLYSLSSVVTVPSSSLGTKTYTCNVDHKPSNT |
| | | KVDKRVESKYGPPCPSCPAPEFLGGPSVFLFPPKPK |
| | | DTLMISRTPEVTCVVVDVSQEDPEVQFNWYVDGVEV |
| | | HNAKTKPREEQFNSTYRVVSVLTVLHQDWLNGKEYK |
| | | CKVSNKGLPSSIEKTISKAKGQPREPQVYTLPPSQEE |
| | | MTKNQVSLTCLVKGFYPSDIAVEWESNGQPENNYKT |
| | | TPPVLDSDGSFFLYSRLTVDKSRWQEGNVFSCSVM |
| | | HEALHNHYTQKSLSLSLGK |
| SEQ ID NO: 12 | Light chain | SYVLTQPPSVSVAPGKTARITCGGNIIGSKLVHWYQQ |
| | | KPGQAPVLVIYDDGDRPSGIPERFSGSNSGNTATLTI |
| | | SRVEAGDEADYYCQVWDTGSDPVVFGGGTKLTVLG |
| | | QPKAAPSVTLFPPSSEELQANKATLVCLISDFYPGAV |
| | | TVAWKADSSPVKAGVETTTPSKQSNNKYAASSYLSL |
| | | TPEQWKSHRSYSCQVTHEGSTVEKTVAPTECS |

Methods for identifying, isolating and testing (e.g. binding and neutralisation) of antibodies and fragment thereof are well-known in the art. See WO 2005/007699, which teaches the identification and characterisation of various anti-IL13 antibodies and fragments and provides suitable methods for doing so.

### Antibody Concentration

The anti-IL-13 antibody may be present in the pharmaceutical composition in any suitable amount. For example, the anti-IL-13 antibody or an IL-13-binding fragment thereof is present at a concentration of 1-300 mg/ml. Preferably, the anti-IL-13 antibody or an IL-13-binding fragment thereof is present at a concentration of 100-200 mg/ml. More preferably, the anti-IL-13 antibody or an IL-13-binding fragment thereof is present at a concentration of 130-170 mg/ml. Most preferably, the anti-IL-13 antibody or an IL-13-binding fragment thereof is present at a concentration of 150 mg/ml±10%, e.g. 150 mg/ml.

### Histidine Buffer

The pharmaceutical compositions of the invention include a histidine buffer. Examples of a histidine buffer include histidine/histidine-HCl, histidine chloride, histidine acetate, histidine phosphate, histidine sulfate, and histidine succinate. In a preferred embodiment, the histidine buffer is histidine/histidine-HCl. Preferably, the histidine buffer is present in an amount of 20 mM±40% (i.e. 12-32 mM), 20 mM±30% (i.e. 14-26 mM), 20 mM±20% (i.e. 16-24 mM), or most preferably 20 mM±10% (i.e. 18-22 mM). In some preferred embodiments, the histidine buffer is present in an amount of 15-25 mM, such as 20 mM.

### Positively Charged Excipients

The pharmaceutical compositions of the invention include a positively-charged excipient. Examples of positively charged excipients include lysine, arginine, sodium chloride (NaCl), glutamate and proline. Preferably, the positively charged excipient is lysine or arginine. Most preferably, the positively charged excipient is lysine. Preferably, the positively charged excipient is present in an amount of 150 mM±40% (i.e. 90-210 mM), 150 mM±30% (i.e. 105-195 mM), 150 mM±20% (120-180 mM), or most preferably 150 mM±10% (i.e. 135-165 mM). In some preferred embodiments, the positively charged excipient is present in an amount of 100-200 mM, such as 150 mM.

### Surfactant

The pharmaceutical compositions of the invention include a surfactant, e.g. a non-ionic surfactant. Examples of surfactants include polysorbate, e.g. polysorbate 20 and polysorbate 80. Preferably, the surfactant is polysorbate-80. Preferably, the pharmaceutical composition comprises 0.001-0.1% (w/w) surfactant, more preferably 0.005-0.05% (w/w) surfactant, and most preferably 0.01% (w/w) surfactant.

pH

Preferably, the pharmaceutical compositions of the invention have a pH of 5.2 to 5.7. More preferably, the pH is 5.5±0.1.

Pharmaceutical Compositions and Formulations Exemplary pharmaceutical compositions comprising the IL-13 antibody Tralokinumab are disclosed in, for example, WO 2007/036745 and WO 2018/158332.

In addition to the specific components disclosed herein, the pharmaceutical compositions of the invention may include any pharmaceutically acceptable excipient.

It will be appreciated that references to “pharmaceutically acceptable excipient” includes references to any excipient conventionally used in pharmaceutical compositions. Such excipients may typically include one or more surfactant, inorganic or organic salt, stabilizer, diluent, solubilizer, reducing agent, antioxidant, chelating agent, preservative and the like.

### Treatment of IL-13 Related Disorder

Generally, the terms “treat”, “treating”, “treatment”, or the like, mean to alleviate (reduce, minimise, or eliminate) symptoms, or to reduce, minimise or eliminate the causation of symptoms either on a temporary or permanent basis.

Preferably, the IL-13 related disorder to be treated is atopic dermatitis, asthma (e.g. allergic asthma), allergic rhinitis, fibrosis, chronic obstructive pulmonary disease, scleroderma, inflammatory bowel disease or Hodgkin's lymphoma. In a preferred embodiment, the IL-13 related disorder is atopic dermatitis, e.g. moderate-to-severe or severe atopic dermatitis (AD).

### Administration

In the methods described herein, the anti-IL-13 antibody or an IL-13-binding fragment thereof may be administered by any appropriate method. Typically, administration is parenteral, e.g. intradermal, intramuscular, intravenous and subcutaneous. Subcutaneous administration is particularly preferred (e.g. as illustrated in the examples). Each dose of the anti-IL-13 antibody or IL-13-binding fragment thereof may therefore be administered subcutaneously.

Administration is preferably in a “therapeutically effective amount”, this being sufficient to show improvement or maintained improvement in one or more disease-associated parameter, patient-related outcome, or achievement of a low disease state.

Administration may be by any convenient route, for example by infusion or bolus injection, by absorption through epithelial or mucocutaneous linings (e.g. oral mucosa, rectal and intestinal mucosa, etc.).

Subcutaneous or intravenous delivery may be with a standard needle and syringe (e.g. including with a prefilled syringe). It is envisaged that the methods described herein will not be restricted to use in the clinic. Therefore, subcutaneous injection using a needle free device is also preferred. Such delivery devices can be reusable or disposable. Numerous reusable pen and autoinjector delivery devices are known in the art and may find use in the present invention. Examples include AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ 1, 11 and 111 (Nova Nordisk, Copenhagen, Denmark), NOVOPEN JUNIOR™ (Nova Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany). Exemplary disposable pen delivery devices for subcutaneous delivery that may find use in the present invention applications include the SOLOSTAR™ pen (Sanofi-Aventis), the FLEXPEN™ (Nova Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park Ill.).

Provided herein is an injector, such as a standard needle and syringe (e.g. a prefilled syringe) or a needle-free injector device (e.g. a pen and autoinjector delivery device), comprising the pharmaceutical composition comprising an anti-IL-13 antibody or IL-13-binding fragment thereof as described herein. In some embodiments the injector contains 2 ml of the pharmaceutical composition comprising an anti-IL-13 antibody or IL-13-binding fragment thereof as described herein. In a preferred embodiment, the injector contains 2 ml of the pharmaceutical composition comprising 300 mg of the anti-IL-13 antibody or IL-13-binding fragment thereof as described herein.

Each dose of anti-IL-13 antibody or IL-13-binding fragment thereof is not necessarily administered in a single administration step (e.g. one injection). Indeed, depending on the concentration of the anti-IL-13 antibody or IL-13-binding fragment thereof (e.g. in the pharmaceutical composition), one, two, three or more administration steps (e.g. one, two, three or more injections) may be required to provide the subject with the required amount of IL-13 binding protein, i.e. anti-IL-13 antibody or IL-13-binding fragment thereof (e.g. a 300 mg dose, for example). Thus, in some embodiments, each dose of the IL-13 binding protein (e.g. of anti-IL-13 antibody or IL-13-binding fragment thereof) is administered in one or two injections (e.g. subcutaneously). Typically subcutaneous injections have a volume of around 1.5 mL or less, such as a volume of from 0.2 to 1.5 mL, e.g. around 1 mL. However, in some embodiments, each dose of the anti-IL-13 antibody or IL-13-binding fragment thereof is administered as a 2 mL injection. For example, in some embodiments, a 300 mg dose of the anti-IL-13 antibody or IL-13-binding fragment thereof is administered as a single 2 mL injection.

### Viscosity

The pharmaceutical compositions of the invention have reduced viscosity, Preferably, the viscosity of the pharmaceutical compositions of the invention is less than 15 centipoise (cP) at 18° C. or 23° C. In some embodiments, the viscosity of the pharmaceutical compositions of the invention is less than 10 centipoise (cP) at 23° C.

### Other Definitions

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

As used herein, the term “sequence identity” or “identity” denotes a property of sequences that measures their similarity or relationship. The term “sequence identity” or “identity” as used in the present disclosure means the percentage of pair-wise identical residues—following (homologous) alignment of a sequence of a protein or polypeptide of the disclosure with a sequence in question—with respect to the number of residues in the longer of these two sequences. Sequence identity is measured by dividing the number of identical amino acid residues by the total number of residues and multiplying the product by 100.

A skilled artisan will recognize available computer programs, for example BLAST (Altschul et al., Nucleic Acids Res, 1997), BLAST2 (Altschul et al., J Mol Biol, 1990), FASTA (which uses the method of Pearson and Lipman (1988)), the TBLASTN program, of Altschul et al. (1990) supra, GAP (Wisconsin GCG package, Accelerys Inc, San Diego USA) and Smith-Waterman (Smith and Waterman, J Mol Biol, 1981), for determining sequence identity using standard parameters. The percentage of sequence identity can, for example, be determined herein using the program BLASTP, version 2.2.5, Nov. 16, 2002 (Altschul et al., Nucleic Acids Res, 1997). In this embodiment, the percentage of homology is based on the alignment of the entire protein or polypeptide sequences (matrix: BLOSUM 62; gap costs: 11.1; cut off value set to 10⁻³) including the polypeptide sequences, preferably using the wild-type protein scaffold as reference in a pairwise comparison. It is calculated as the percentage of numbers of “positives” (homologous amino acids) indicated as result in the BLASTP program output divided by the total number of amino acids selected by the program for the alignment. Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps, which are spaces in an alignment that are the result of additions or deletions of amino acids. Generally, default parameters are used, with a gap creation penalty equaling 12 and a gap extension penalty equaling 4.

Specifically, in order to determine whether an amino acid residue of the amino acid sequence of an anti-IL-13 antibody or a fragment thereof is different from another antibody sequence, a skilled artisan can use means and methods well-known in the art, e.g., alignments, either manually or by using computer programs such as BLAST 2.0, which stands for Basic Local Alignment Search Tool, or ClustalW, or any other suitable program which is suitable to generate sequence alignments.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

All publications mentioned herein are incorporated by reference in their entirety.

### EXAMPLES

### Example 1: Development of a Low Viscosity Formulation

Tralokinumab may be formulated with 50 mM sodium acetate/acetic acid, 85 mM sodium chloride, and 0.01% w/v polysorbate 80, at pH 5.5 (Formulation A). Such a formulation was disclosed in, for example, WO 2007/036745.

Five Tralokinumab formulations were tested against Formulation A to assess their viscosities. The tested formulations include (i) 150 mM proline and 100 mM sucrose, (ii) 150 mM glutamate, (iii) 150 mM NaCl, (iv) 150 mM arginine or (v) 150 mM lysine. Viscosity was measured over a range of temperatures.

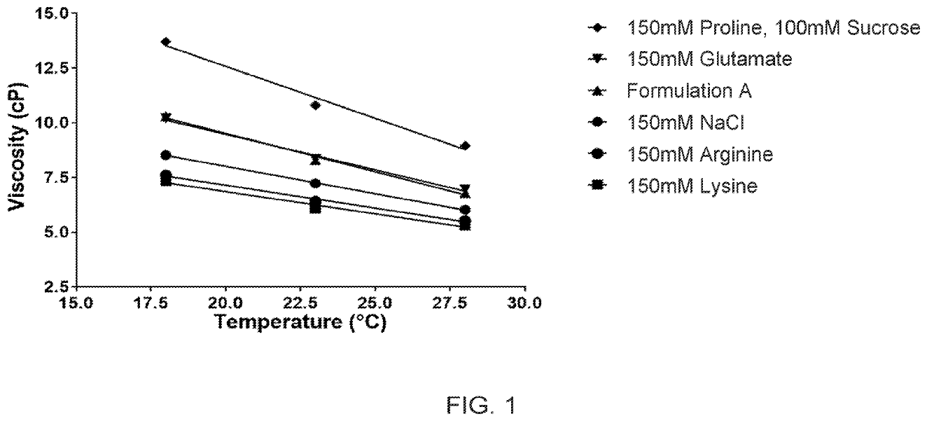

FIG. 1 shows the viscosity of Formulation A against the new formulations at a range of temperatures. Addition of positively charged excipients, such as lysine and arginine, dramatically decreased the viscosity of the drug product. Addition of NaCl also resulted in a decrease in viscosity compared to Formulation A. Formulations including glutamate showed a similar viscosity to Formulation A and addition of proline and sucrose resulted in increased viscosity compared to Formulation A.

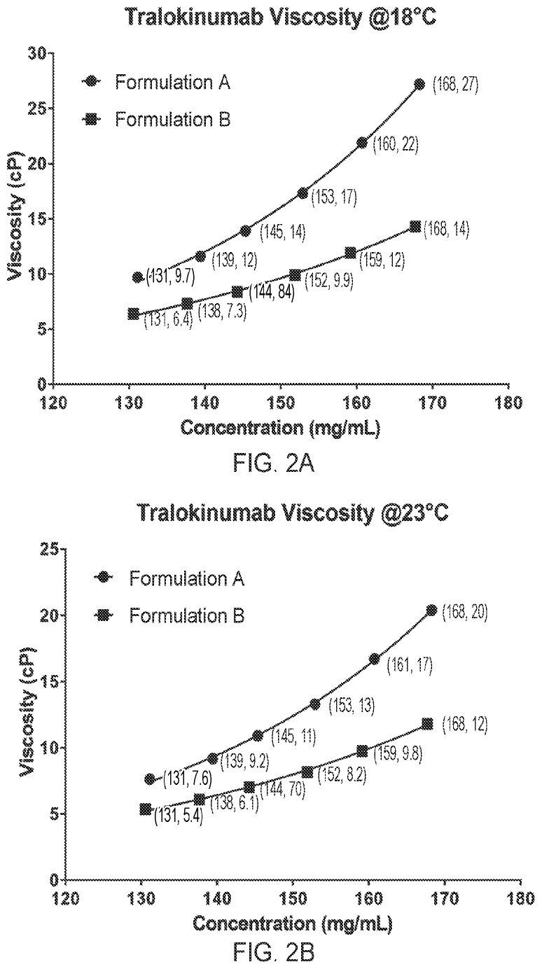

A low viscosity formulation selected for further analysis comprised Tralokinumab with 20 mM histidine/histidine HCl, 150 mM lysine and 0.01% w/v polysorbate 80, at pH 5.5 (Formulation B). The target concentration for use of Tralokinumab in Formulation A is 150 mg/ml and the viscosity of Formulation B was compared to Formulation A at concentrations of between 130-170 mg/mL Tralokinumab. Viscosity was measured at both 18° C. and 23° C. Formulation B showed less of a difference in viscosity from the lower to the upper end of the concentration specification (±10% of 150 mg/mL) at both temperatures measured, compared to Formulation A (see FIGS. 2A and B).

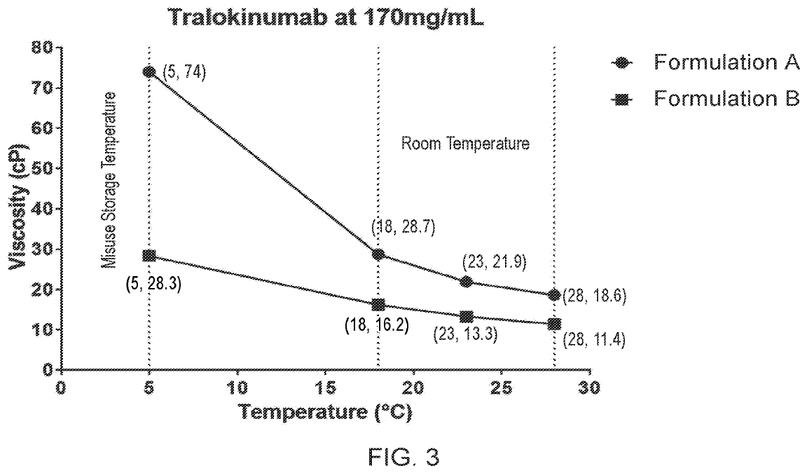

The viscosities of Formulations A and B with Tralokinumab concentrations of 170 mg/ml were also measured at various temperatures between 5-30° C. Formulation B showed less susceptibility to viscosity changes with temperature than Formulation A and this was demonstrated most dramatically at 5° C. (see FIG. 3).

### Example 2: Challenges of High Viscosity to Drug Delivery

The high injection forces required to deliver a viscous product from a 2 mL prefilled syringe (PFS) leads to concerns around patient tolerability and technical challenges for autoinjector development. The infusion duration window is also broader for a viscous formulation in a large volume bolus injector (LVBI).

In a given syringe and needle, flow rate and solution viscosity are the only two parameters that can be controlled and are proportional to force:

$$F = ( 8 {QDIR}_{s}^{2} η ) / {R}_{n}^{4} + {F}_{friction}$$

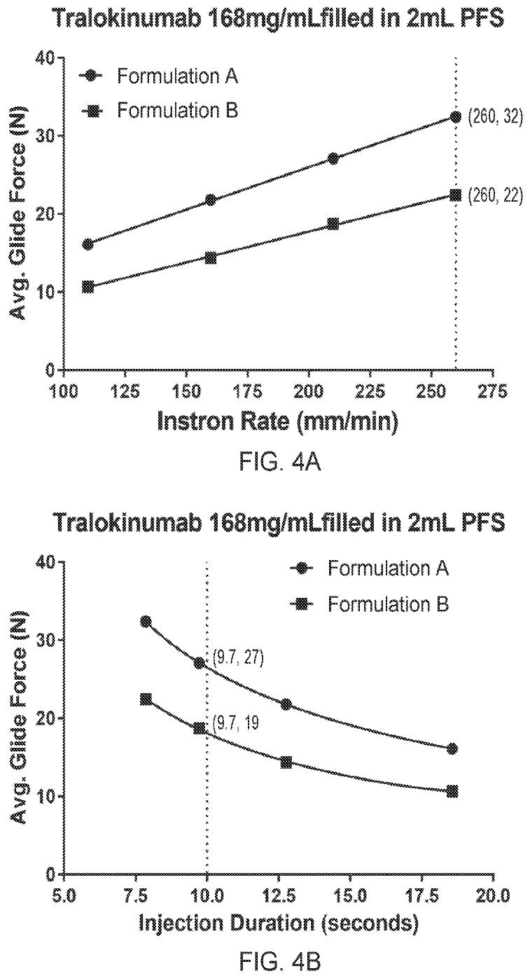

The force required to expel Formulations A or B from a 2 mL PFS was measured for varying instron rates (see FIG. 4A). In this experiment, a Tralokinumab concentration of 168 mg/mL was used. Results show that Formulation B enables the injection to be delivered in a shorter time frame using less force compared to Formulation A (see FIG. 4B). This would in turn lead to a more comfortable injection.

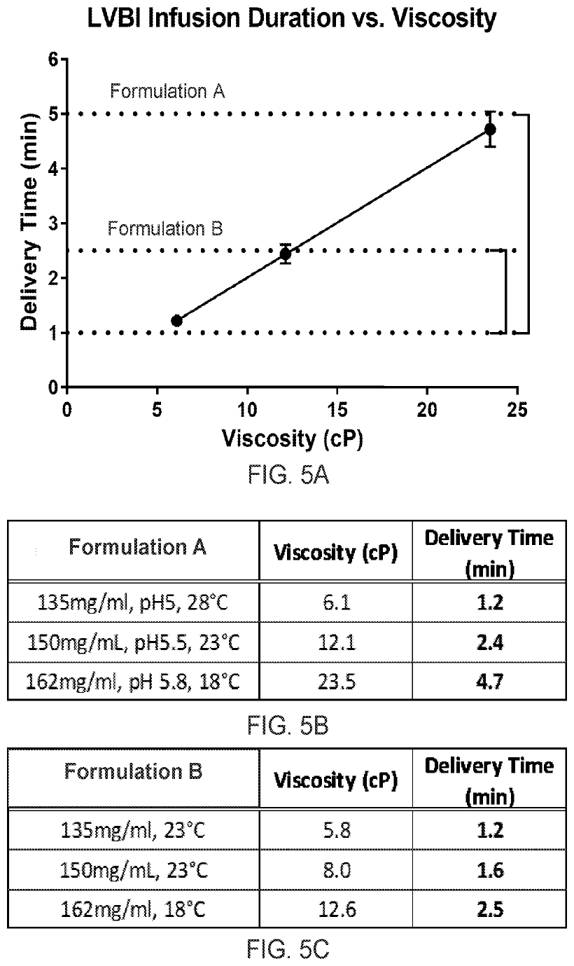

The effect of lowering viscosity on the delivery time of LVBI was also assessed. As shown in FIG. 5A, lowering the viscosity shortens the infusion duration. Lowering the viscosity may therefore increase patient convenience and compliance. The viscosity and delivery times of various concentrations of Formulations A and B are shown in FIGS. 5B and C. The delivery times are lower for Formulation B compared to Formulation A.

### Example 3: Stability of Low Viscosity Formulation

Additional advantages of Formulation B, such as stability, were also assessed.

The stability of Formulation B in a 1 mL PFS was compared with Formulation A at both 5° C. and 25° C. A Tralokinumab concentration of 168 mg/mL was used in this experiment. Purity was measured at various time points by size exclusion chromatography (SEC).

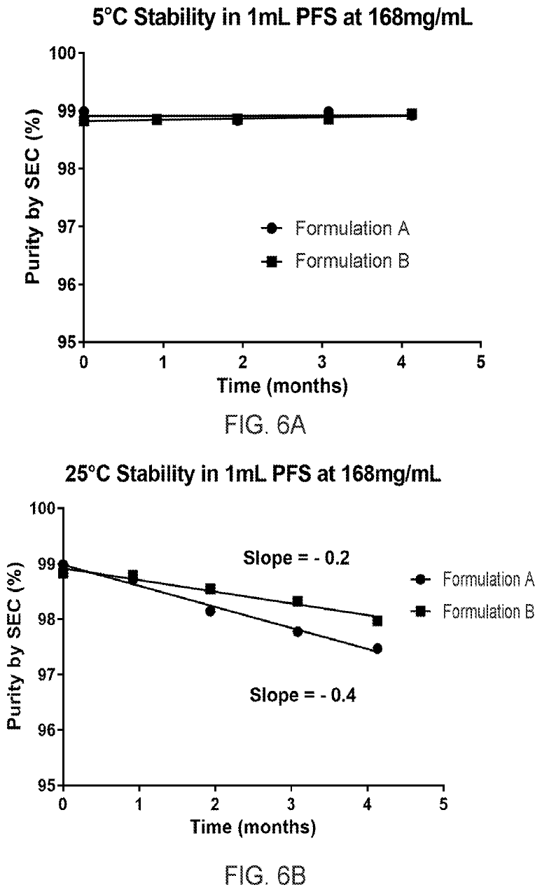

After 4 months, the stability of Formulation B was equivalent (5° C.) or superior (25° C.) to that of Formulation A (see FIGS. 6A and B).

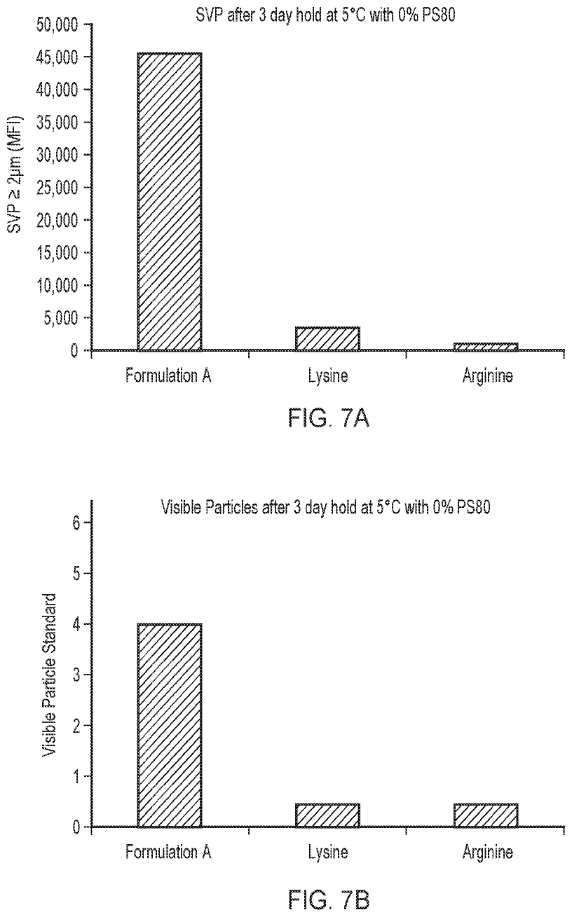

The levels of subvisible or visible particles in Formulation A after 3 days at 5° C. without any polysorbate-80 were measured and the effect of the addition of lysine or arginine to this formulation was assessed. Results showed that addition of either lysine or arginine substantially lowered both subvisible and visible particles in the pre-formulated bulk (see FIGS. 7A and B).

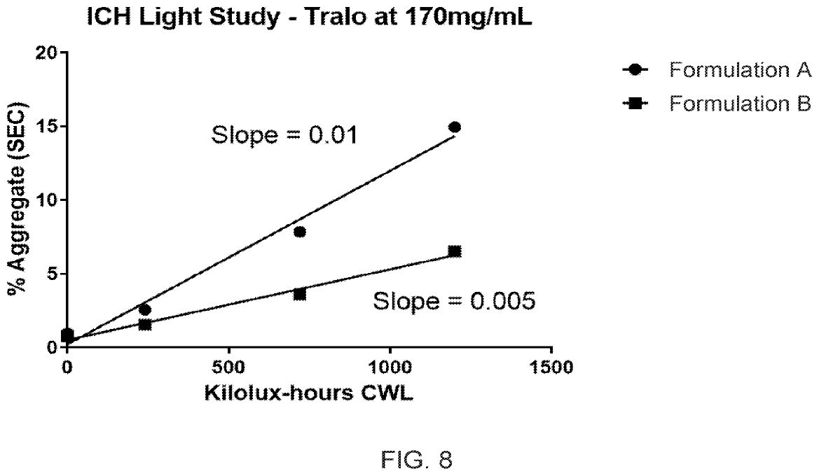

The sensitivity of Formulation B to light exposure was also compared to that of Formulation A. Here, the formulations comprised 170 mg/mL Tralokinumab. Results showed that Formulation B was substantially less sensitive to light exposure than Formulation A (see FIG. 8).

### CONCLUSIONS

The examples demonstrate the development of a low viscosity Tralokinumab formulation (Formulation B), which has a significant beneficial impact for drug delivery in the various device options and additional advantages to stability and manufacturing, as well as reduced sensitivity to light.

### REFERENCES

A number of publications are cited above in order to more fully describe and disclose the invention and the state of the art to which the invention pertains. Full citations for these references are provided below. The entirety of each of these references is incorporated herein.
Wills-Karp, M. et al., Science, 1998. 282 (5397), 2258-2261
Grunig, G. et al., Science, 1998. 282 (5397), 2261-2263
Venkayya, R., et al., Am J Respir Cell Mol Biol, 2002. 26 (2), 202-208
Morse, B. et al., Am J Physiol Lung Cell Mol Physiol, 2002. 282 (1), L44-49
Zhu, Z. et al., J Clin Invest, 1999. 103 (6), 779-788
Nomura, I., Goleva, E., Howell, M. D., Hamid, Q. A., Ong, P. Y., Hall, C. F. et al. Cytokine milieu of atopic dermatitis, as compared to psoriasis, skin prevents induction of innate immune response genes. J Immunol. 2003; 171:3262-3269
Tazawa, T., Sugiura, H., Sugiura, Y., and Uehara, M. Relative importance of IL-4 and IL-13 in lesional skin of atopic dermatitis. Arch Dermatol Res. 2004; 295:459-464
van der Pouw Kraan, T. C. et al., Genes Immun, 1999. 1 (1), 61-65
Hasegawa, M. et al., J Rheumatol, 1997. 24 (2), 328-332
Hancock, A. et al., Am J Respir Cell Mol Biol, 1998. 18 (1), 60-65
Zheng, T. et al., J Clin Invest, 2000. 106 (9), 1081-1093
Heller, F. et al., Immunity, 2002. 17 (5), 629-38
Fiumara, P. et al., Blood, 2001. 98 (9), 2877-2878
Terabe, M. et al., Nat Immunol, 2000. 1 (6), 515-520
Ahlers, J. D. et al., Proc Natl Acad Sci US A, 2002. 99 (20), 13020-13025
Popovic et al. J. Mol. Biol. (2017) 429 (2): 208-219
May, R. D., Monk, P. D., Cohen, E. S., Manuel, D., Dempsey, F., Davis, N. H. et al. Preclinical development of CAT-354, an IL-13 neutralizing antibody, for the treatment of severe uncontrolled asthma. Br J Pharmacol. 2012; 166:177-193
Wollenberg J. Allergy Clin. Immunol. (2019) 143 (1): 135-141
“International Nonproprietary Names for Pharmaceutical Substances (INN)” list 102 (WHO Drug Information (2009) 23 (4): pp 348
Heinzmann, A. et al., Hum Mol Genet, 2000. 9 (4), 549-559
Howard, T. D. et al., Am J Hum Genet, 2002. 70 (1), 230-236
Kauppi, P. et al., Genomics, 2001. 77 (1-2), 35-42
Graves, P. E. et al., J Allergy Clin Immunol, 2000. 105 (3), 506-513

1. A pharmaceutical composition comprising an anti-IL-13 antibody or an IL-13-binding fragment thereof, 15-25 mM histidine buffer, 100-200 mM positively charged excipient (e.g. lysine or arginine), 0.01% polysorbate 80, pH 5.5±0.1, wherein the anti-IL-13 antibody or IL-13-binding fragment thereof comprises a heavy chain variable region (HCVR) and a light chain variable region (LCVR), wherein:
(i) the heavy chain variable region comprises:
a heavy chain complementarity determining region 1 (HCDR1) comprising an amino acid sequence of SEQ ID NO:1;
a heavy chain complementarity determining region 2 (HCDR2) comprising an amino acid sequence of SEQ ID NO:2; and
a heavy chain complementarity determining region 3 (HCDR3) comprising an amino acid sequence of SEQ ID NO:3; and
(ii) the light chain variable region comprises:
a light chain complementarity determining region 1 (LCDR1) comprising an amino acid sequence of SEQ ID NO:4;
a light chain complementarity determining region 2 (LCDR2) comprising an amino acid sequence of SEQ ID NO:5; and
a light chain complementarity determining region 3 (LCDR3) comprising an amino acid sequence of SEQ ID NO:6.

2. The pharmaceutical composition of claim 1 or 2, wherein the anti-IL-13 antibody or IL-13-binding fragment thereof is present at a concentration of between 130 and 170 mg/ml.

3. The pharmaceutical composition of claim 3, wherein the anti-IL-13 antibody or IL-13-binding fragment thereof is present at a concentration of 150 mg/ml±10%.

4. The pharmaceutical composition according to any one of the preceding claims, wherein the histidine buffer is present in an amount of 20 mM±10%.

5. The pharmaceutical composition according to any one of the preceding claims, wherein the positively charged excipient is lysine.

6. The pharmaceutical composition according to any one of the preceding claims, wherein the positively charged excipient is present in an amount of 150 mM±10%.

7. The pharmaceutical composition according to any one of the preceding claims, wherein the anti-IL-13 antibody is a human IL-13 monoclonal antibody or an IL-13-binding fragment thereof.

8. The pharmaceutical composition according to any one of the preceding claims, wherein the anti-IL-13 antibody is an IgG4 antibody or an IL-13-binding fragment thereof.

9. The pharmaceutical composition according to any one of the preceding claims, wherein the IL-13-binding fragment is selected from a Fab, Fab′, F (ab′) 2, Fd, Fv, single-chain Fv (scFv), or disulfide-linked Fvs (sdFv).

10. The pharmaceutical composition according to any one of the preceding claims, wherein the anti-IL-13 antibody or IL-13-binding fragment thereof comprises:
(i) an amino acid sequence that is at least 80%, at least 85%, at least 90%, at least 95%, at least 96%, at least 97%, at least 98%, or at least 99% identical to a heavy chain variable region sequence of SEQ ID NO: 8; and/or
(ii) an amino acid sequence that is at least 80%, at least 85%, at least 90%, at least 95%, at least 96%, at least 97%, at least 98%, or at least 99% identical to a light chain variable region sequence of SEQ ID NO: 10.

11. The pharmaceutical composition according to claim 10, wherein the anti-IL-13 antibody or IL-13-binding fragment thereof comprises a heavy chain variable region sequence of SEQ ID NO: 8 and a light chain variable region sequence of SEQ ID NO: 10.

12. The pharmaceutical composition according to any one of the preceding claims, wherein the anti-IL-13 antibody or IL-13-binding fragment thereof comprises:
(i) an amino acid sequence that is at least 80%, at least 85%, at least 90%, at least 95%, at least 96%, at least 97%, at least 98%, or at least 99% identical to the heavy chain sequence of SEQ ID NO: 11; and/or
(ii) an amino acid sequence that is at least 80%, at least 85%, at least 90%, at least 95%, at least 96%, at least 97%, at least 98%, or at least 99% identical to the light chain sequence of SEQ ID NO: 12.

13. The pharmaceutical composition according to claim 12, wherein the anti-IL-13 antibody or IL-13-binding fragment thereof comprises a heavy chain sequence of SEQ ID NO: 11 and a light chain sequence of SEQ ID NO: 12.

14. The pharmaceutical composition according to any one of the preceding claims, wherein the anti-IL-13 antibody is Tralokinumab.

15. An injector containing the pharmaceutical composition according to any one of the preceding claims.

16. The injector according to claim 15, wherein the injector contains 2 ml of the pharmaceutical composition of any one of claims 1 to 14.

17. The injector according to claim 15 or 16, wherein the pharmaceutical composition comprises 300 mg of the anti-IL-13 antibody or IL-13-binding fragment thereof.

18. The pharmaceutical composition according to any one of claims 1 to 14 for use in a method for treatment of an IL-13 related disorder.

19. The pharmaceutical composition for use according to claim 18, wherein the IL-13 related disorder is selected from atopic dermatitis, asthma, allergic rhinitis, fibrosis, chronic obstructive pulmonary disease, scleroderma, inflammatory bowel disease and Hodgkin's lymphoma.

20. The pharmaceutical composition for use according to claim 19, wherein the IL-13 related disorder is atopic dermatitis.

21. A method for the treatment of an IL-13 related disorder, wherein the method comprises the step of administering the pharmaceutical composition according to any one of claims 1 to 14 in a therapeutically effective amount to a patient in need thereof.

22. The method according to claim 21, wherein the IL-13 related disorder is selected from atopic dermatitis, asthma, allergic rhinitis, fibrosis, chronic obstructive pulmonary disease, scleroderma, inflammatory bowel disease and Hodgkin's lymphoma.

23. The method according to claim 22, wherein the IL-13 related disorder is atopic dermatitis.

24. The pharmaceutical composition for use according to any one of claims 18-20 or the method of any one of claims 21-23, wherein the method for treatment comprises administering a 300 mg dose of the anti-IL-13 antibody or IL-13-binding fragment thereof as a single 2 ml injection.


SEQUENCE LISTING (12 sequences)

SEQ ID NO: 1 - protein, 5 aa, synthetic construct
  REGION 1..5: note = HCDR1
NYGLS

SEQ ID NO: 2 - protein, 17 aa, synthetic construct
  REGION 1..17: note = HCDR2
WISANNGDTNYGQEFQG

SEQ ID NO: 3 - protein, 13 aa, synthetic construct
  REGION 1..13: note = HCDR3
DSSSSWARWFFDL

SEQ ID NO: 4 - protein, 11 aa, synthetic construct
  REGION 1..11: note = LCDR1
GGNIIGSKLVH

SEQ ID NO: 5 - protein, 7 aa, synthetic construct
  REGION 1..7: note = LCDR2
DDGDRPS

SEQ ID NO: 6 - protein, 11 aa, synthetic construct
  REGION 1..11: note = LCDR3
QVWDTGSDPVV

SEQ ID NO: 7 - DNA, 366 bp, synthetic construct
  misc_feature 1..366: note = cDNA heavy chain variable domain
  source 1..366: mol_type = other DNA
caggtccagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcagcgctaataatggcgacacaaattatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 8 - protein, 122 aa, synthetic construct
  REGION 1..122: note = Polypeptide sequence heavy chain variable region
QVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSS

SEQ ID NO: 9 - DNA, 327 bp, synthetic construct
  misc_feature 1..327: note = cDNA light chain variable domain
  source 1..327: mol_type = other DNA
tcctatgtgctgactcagccaccctcggtgtcagtggccccaggaaagacggccaggattacctgtgggggaaacatcattggaagtaaacttgtacactggtaccagcagaagccaggccaggcccctgtgctggtcatctatgatgatggcgaccggccctcagggatccctgagcgattctctggctccaactctgggaacacggccaccctgaccatcagcagggtcgaggccggggatgaggccgactattattgtcaggtgtgggatactggtagtgatcccgtggtattcggcggagggaccaagctgaccgtcctaggt

SEQ ID NO: 10 - protein, 108 aa, synthetic construct
  REGION 1..108: note = Polypeptide sequence light chain variable region
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVL

SEQ ID NO: 11 - protein, 449 aa, synthetic construct
  REGION 1..449: note = Heavy chain
QVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 214 aa, synthetic construct
  REGION 1..214: note = Light chain
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS